(19) United States
(12) Patent Application Publication
(10) Pub. No.: US 2021/0118059 A1
(43) Pub. Date: Apr. 22, 2021

(54) STEP-COUNT MANAGEMENT APPARATUS

(71) Applicant: Y4.com Co., Ltd, Fukuoka-shi, Fukuoka (JP)

(72) Inventor: Yukinao AJIMA, Fukuoka-shi, Fukuoka (JP)

(21) Appl. No.: 16/981,880

(22) PCT Filed: Feb. 6, 2019
(86) PCT No.: PCT/JP2019/004219

§ 371 Date: Sep. 17, 2020

(30) Foreign Application Priority Data
Mar. 21, 2018 (JP) 2018-053593

(51) Int. Cl. G06Q 40/08; G06Q 40/02; G06Q 30/02; G16H 15/00; G06Q 10/10; G06Q 50/26; G06Q 50/22; G06Q 20/38; G16H 10/65; G16H 20/30; A63B 24/00; G01C 22/00

(52) U.S. Cl. CPC G06Q 40/08; G06Q 40/02; G06Q 30/0284; G16H 15/00; G06Q 10/10; G06Q 50/26; A63B 2220/17; G06Q 20/381; G16H 10/65; G16H 20/30; A63B 24/0062; G01C 22/006; G06Q 50/22

(57) ABSTRACT

[Problem] To allow a person who performs the act of walking to receive a certain amount of money commensurate with the number of steps the person makes, thereby motivating the person to perform the act of walking and facilitating medical cost reduction.

[Solution] A step-count management apparatus includes: a means configured to store the number of steps measured by a pedometer; a means configured to store the balance of electronic money owned by the user; a means configured to compute first information that indicates a medical cost reduction effect by multiplying the number of steps by a first coefficient that indicates a medical cost reduction effect per step; a means configured to report the first information to a health insurance server device; a means configured to acquire, from the health insurance server device, second information that can be cashed and is computed by multiplying the first information by a second coefficient for computing the second information; a means configured to add an amount of money that is computed by multiplying the second information by a third coefficient for converting the second information into electronic money, to the balance of electronic money; and a means configured to request the health insurance server device to cash the second information that has been convrted into electronic money.

### FIELD

The present invention relates to a technique for converting step-count information that is measured by a pedometer into information that has a monetary value.

### BACKGROUND

In recent years, social security related expenses (medical expenses) have increased in this country, so that government finances are apparently put under pressure by these expenses. To overcome this situation, it becomes more important to create a system to reduce medical costs through individual efforts.

Meanwhile, companies, local governments, etc. recommend walking due to the fact that walking is effective in maintaining health and decreasing obesity. It is widely known that proper exercise is effective in preventing obesity, high blood pressure and so forth, and physical activity promotion is now an important policy from the perspective of curbing medical expenses.

In such a situation, for example, Patent Document 1 proposes an attempt to link the number of steps measured by a pedometer to health promotion. Further, Non-Patent Document 1 publicizes the content that the walking action itself has a certain monetary effect of curbing medical expenses.

### RELATED ART DOCUMENTS

### Patent Documents

Patent Document 1: Japanese Patent Number 6168374

### Non-Patent Documents

Non-Patent Document 1: Expected to decrease medical costs by developing a community where people can enjoy daily life on foot—“Guideline for surveying the amount of walking (number of steps) to assess the effectiveness of promoting health in community development”
Ministry of Land, Infrastructure, Transport and Tourism Mar. 10, 2017 (http://www.mlit.go.jp/report/press/toshi07_hh_000107.html)

### SUMMARY

### Technical Problem

Although Non-Patent Document 1 indicates that medical costs can be reduced by walking, there is a problem that no system allows a person who perform the act of walking to directly receive a certain amount of money commensurate with the expected reduction costs.

In view of the above problem, the purpose of the present invention is to provide a step-count management apparatus, which allows a person who performs the act of walking to receive a certain amount of money commensurate with the number of steps the person makes, thereby motivating the person to perform the act of walking and facilitating medical cost reduction.

### Solution to Problem

An aspect of a step-count management apparatus to be disclosed is characterized by comprising: a step-count memory configured to store the number of steps measured by a pedometer that counts the number of steps a user makes; an electronic money balance memory configured to store the balance of electronic money owned by the user; a first information computation means configured to compute first information by multiplying the number of steps by a first coefficient that indicates a medical cost reduction effect per step, the first information indicating a medical cost reduction effect based on the number of steps the user makes; a first information reporting means configured to report the first information to a health insurance server device that is managed by an organization that provides insurance benefits covered by public medical insurance; a second information acquisition means configured to acquire, from the health insurance server device, second information that can be cashed by the organization, the health insurance server device multiplying the first information by a second coefficient to thereby compute the second information based on the medical costs expected to be reduced due to the user's act of walking; an electronic money conversion means configured to add an amount of money that is computed by multiplying the second information by a third coefficient for converting the second information into electronic money, to the balance of electronic money; and a cashing request. means configured to request the health insurance server device to cash the second information that has been converted into electronic money.

Another aspect of the step-count management apparatus to be disclosed is characterized by comprising: a step-count memory configured to store the number of steps measured by a pedometer that counts the number of steps a user makes; an electronic money balance memory configured to store the balance of electronic money owned by the user; a step-count reporting means configured to report the number of steps to a health insurance server device that is managed by an organization that provides insurance benefits covered by public medical insurance; a second information acquisition means configured to acquire, from the health insurance server device, second information that can be cashed by the organization, the health insurance server device multiplying the number of steps by a second coefficient to thereby compute the second information based on the medical costs expected to be reduced due to the user's act of walking; an electronic money conversion means configured to add an amount of money that is computed by multiplying the second information by a third coefficient for converting the second information into electronic money, to the balance of electronic money; and a cashing request means configured to request the health insurance server device to cash the second information that has been converted into electronic money.

### EFFECTS

A step-count management apparatus to be disclosed allows a person who performs the act of walking to receive a certain amount of money commensurate with the number of steps the person makes, thereby motivating the person to perform the act of walking and facilitating medical cost reduction.

### BRIEF DESCRIPTION OF THE DRAWINGS

FIG. 1 schematically illustrates a step-count management apparatus according to the present embodiment;

FIG. 2 is a functional block diagram of the step-count management apparatus according to the present embodiment;

FIG. 3 illustrates an example of a hardware structure according to the present embodiment;

FIG. 4 is a flowchart illustrating the flow of a first processing example executed by the step-count management apparatus according to the present embodiment; and

FIG. 5 is a flowchart illustrating the flow of a second processing example executed by the step-count management apparatus according to the present embodiment.

### DETAILED DESCRIPTION

An embodiment for carrying out the present invention will be described with reference to the drawings.

### (The Principle of Operation of the Step-Count Apparatus According to the Present Embodiment)

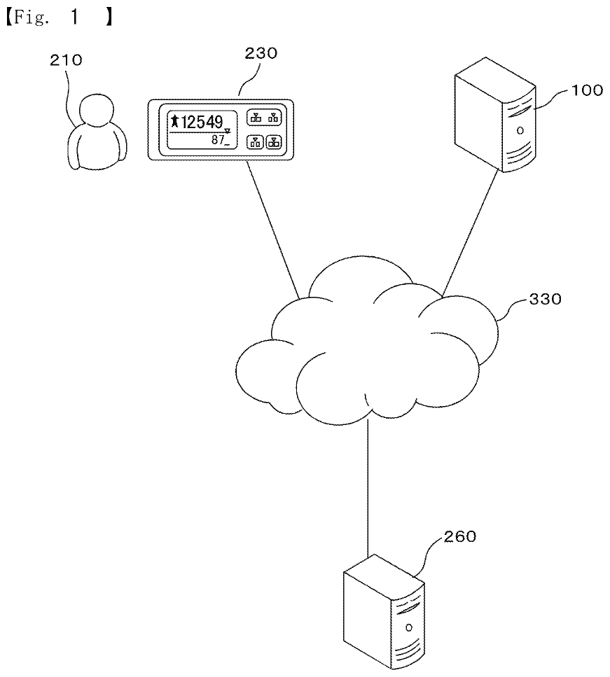

A step-count management apparatus 100 according to the present embodiment (hereinafter, simply referred to as “the present apparatus”) is schematically described with reference to FIG. 1. FIG. 1 illustrates how the present apparatus 100 and peripheral devices 230, 260 are related.

As shown in FIG. 1, the present apparatus 100 is connected, through a communication network 330, to a pedometer 230 operated by a user 210 and a health insurance server device 260 that is managed by an organization 340 that provides insurance benefits covered by public medical insurance that includes employee health insurance and community health insurance. The communication network 330 can be wired or wireless. The pedometer 230 a tool for measuring the number of steps of the user 210. The organization 340 that provides insurance benefits covered by public medical insurance, may be the national health insurance association, each health insurance association, mutual aid association and so forth for employee health insurance, and may be each local government for the national health insurance.

The present apparatus 100 acquires the number of steps 220 a user 210 makes from the pedometer 230 through the communication network 330 and reports to the health insurance server device 260 the number of steps 220 or first information 270 that is computed by multiplying the number of steps 220 by a coefficient 290 that indicates a medical cost reduction effect per step. The health insurance server device 260 computes second information 280 that can be cashed by the organization 340, and reports the second information 280 that was computed to the present apparatus 100. The second information 280 is computed by multiplying the number of steps 220 that was acquired or the first information 270 by a coefficient 300 for computing the second information 280.

The present apparatus 100 adds the information that is computed by multiplying the second information 280 that was acquired by a coefficient 310 for converting the second information 280 into electronic money 240, to a balance 250 of electronic money owned by the user 210. In addition, the present apparatus 100 requests the health insurance server device 260 (organization 340) to pay the amount of cash commensurate with the second information 280 that was converted into the electronic money 240.

As described above, the user 210 can obtain the electronic money 240 on the basis of the act of walking, and therefore the user 210 is highly motivated for the act of walking. As it has already been indicated that the act of walking has some effectiveness in reducing medical costs, the present apparatus 100 allows the user 210 who performs the act of walking to obtain a certain amount of money commensurate with the number of steps, thereby motivating the user 210 to perform the act of walking and facilitating medical cost reduction.

There already exists a system which allows customers who purchase a product or a service to acquire electronic money (including sales promotion points issued by a company). Whereas, the system created by the present apparatus 100 provides a user 210 with the electronic money 240 on the basis of the amount of walking the user 210 does, and the electronic money 240 provided is based on the medical costs that is expected to be reduced due to the act of walking the user 210 does. Therefore, the system created by the present apparatus 100 is fundamentally different from the conventional system.

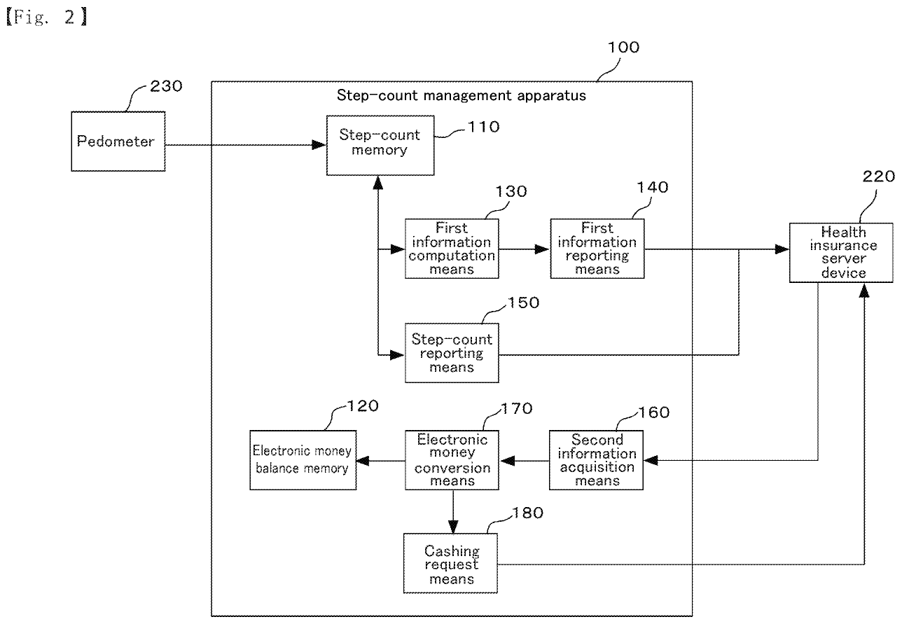

The principle of operation of the present apparatus 100 is now described with reference to FIG. 2. FIG. 2 is a functional block diagram of the present apparatus 100. As shown in FIG. 2, the present apparatus 100 comprises: a step-count memory 110; an electronic money balance memory 120; a first information computation means 130; a first information reporting means 140; a step-count reporting means 150; a second information acquisition means 160; an electronic money conversion means 170; and a cashing request means 180.

The step-count memory 110 stores number of steps 220 measured by a pedometer 230 that counts the number of steps a user 210 makes. In addition, the step-count memory 110 may associate and store the number of steps 220 and measurement environment identification information 320 that is detected when the pedometer 230 measures the number of steps 220, the measurement environment identification information 320 indicating the environment in which the number of steps 220 is measured. The measurement environment identification information 320 is made up of any one or more pieces of information that include the tilt of the ground where the user 210 is located (the user 210 walks), the feature of action taken by the user such as the number of repetitive actions, a type of shoes worn by the user 210 (sneaker, leather shoes, boots, sandals and so forth), the strength of exercise taken by the user 210, and the type and characteristics of the pedometer 230. The measurement environment identification information 320 is used to correct the number of steps counted by the pedometer 230, which allows the pedometer 230 to handle the measured number of steps 220 uniformly irrespective of measurement environment.

The electronic money balance memory 120 stores the balance 250 of electronic money 240 owned by the user 210. The electronic money 240 may be, for example, Edy (registered trademark), but not limited to this type. The electronic money 240 may be virtual currency (not limited to one type).

The first information computation means 130 multiples the number of steps by a first coefficient 290 that indicates a medical cost reduction effect per step, to thus compute the first information 270 that indicates a medical cost reduction effect commensurate with the number of steps 220 the user 210 makes. For example, the medical cost reduction effect per step per day can be calculated as 0.065 to 0.072 Yen, which has been publicized by the Ministry of Land, Infrastructure, Transport and Tourism, on http://www.mlit.go.jp/report/press/toshi07_hh_000107.html.

The first information computation means 130 may compute the first information 270 by multiplying the number of steps 220 corrected based on the measurement environment identification information 320 by the first coefficient 290. The correction based on the measurement environment identification information 320 includes correcting the number of steps 220 to become greater as the tilt of the ground increases from the viewpoint of exercise intensity, correcting the number of steps 220 to become smaller as the number of repetitive actions increases, and correcting the number of steps to become greater as exercise intensity increases. This is because it can be assumed that the medical cost reduction effect increases, as the exercise intensity of the user 210 becomes greater.

The first information reporting means 140 reports the first information 270 to the health insurance server device 260 that is managed by the organization 340 that provides insurance benefits covered by the public medical insurance that includes employee health insurance and community health insurance.

The step-count reporting means 150 reports the number of steps 220 to the health insurance server device 260. Additionally, the step-count reporting means 150 may have a configuration in which the number of steps 220 corrected based on the measurement environment identification information 320 is reported to the health insurance server device 260.

Here, the health insurance server device 260 multiplies the first information 270 reported by the first information reporting means 140 by a second coefficient 300 for computing the second information 280 that can be cashed by the organization 340 on the basis of the first information 270, to thereby compute the second information 280.

The health insurance server device 260 may compute the second information 280 by multiplying the number of steps 220 reported by the step-count reporting means 150 by the second coefficient 300 for computing the second information 280 that can be cashed by the organization 340 on the basis of the number of steps 220. The second information acquisition means 160 acquires the second information 280 computed by the health insurance server device 260 from the health insurance server device 260.

The electronic money conversion means 170 adds an amount of money computed by multiplying the second information 280 by a third coefficient 310 for converting the second information 280 into the electronic money 240, to the balance 250. The third coefficient 310 may be determined by considering the profit of the operator of the present apparatus 100.

The cashing request means 180 requests the health insurance server device 260 to cash the second information 280 that has been converted into the electronic money 240. This is a process for making up for the amount of money that has been paid out to the user 210 as the electronic money 240 by the operator of the present apparatus 100.

As described above, the present apparatus 100 provides the user 210 with the electronic money 240 on the basis of the amount of walking the user 210 does. That is, the user 210 can obtain the electronic money 240 on the basis of the act of walking, and as a result the user 210 is highly motivated for the act of walking. Meanwhile, as it has already been indicated that the act of walking has some effectiveness in reducing medical costs, the present apparatus 100 allows the user 210 who performs the act of walking to obtain a certain amount of money commensurate with the number of steps, thereby motivating the user 210 to perform the act of walking and facilitating medical cost reduction.

### (Hardware Structure of Step-Count Management Apparatus According to the Present Embodiment)

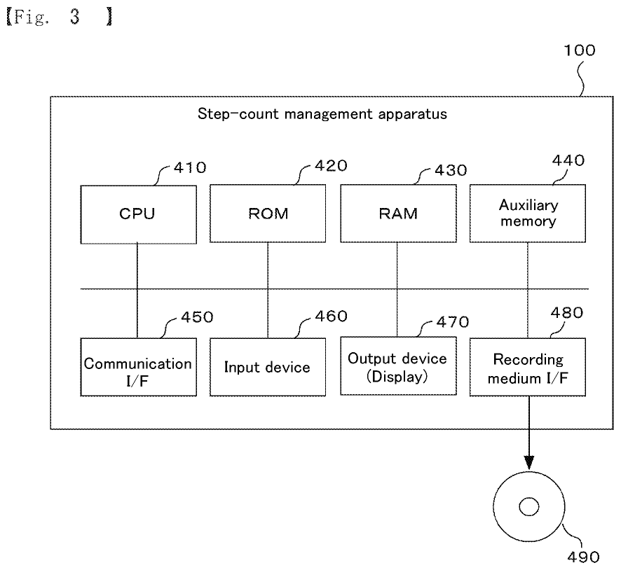

An example of the hardware structure of the present apparatus 100 is now described with reference to FIG. 3. FIG. 3 illustrates an example of the hardware structure according to the present apparatus 100. As shown in FIG. 3, the present apparatus 100 comprises: a central processing unit (CPU) 410, a read only memory (ROM) 420, a random access memory (RAM) 430, an auxiliary memory 440, a communication I/F 450, an input device 460, an output device 470, and a recording medium I/F 480.

The CPU 410 executes a program stored in the ROM 420; the CPU 410 processes data that is loaded in the RAM 430 in accordance with the instructions of the program, to thus control the entire present apparatus 100. The ROM 420 stores the program executed by the CPU 410 and data. The RAM 430 is loaded with data and a program that is executed when the CPU 410 executes the program stored in the RUM 420; the RAM 430 temporarily stores operation data while the CPU 410 executes processing.

The auxiliary memory 440 stores an operation system (OS) and an application program according to the present embodiment and so forth, together with data related to the present embodiment. The auxiliary memory 440 includes the step-count memory 110 and the electronic money balance memory 120; the auxiliary memory 440 is made up of, for example, a hard disc drive (HDD) and a flash memory.

The communication I/F 450 is connected to the communication network 330 such as a wired or wireless local area network (LAN) and the internet; the communication I/F 450 serves as an interface that sends and receives data to and from other devices 230, 260 equipped with communication functions.

The input device 460 is, for example, a keyboard that is used to input data into the present apparatus 100. The output device (display) 470 is made up of a liquid crystal display (LCD) and so forth; the output device 470 serves as a user interface when the user uses functions provided by the present apparatus 100 or makes various settings. The recording medium I/F 480 serves as an interface to send or receive data to or from a recording medium 490 such as a CD-ROM, a DVD-ROM, a USB memory and so forth.

Each means included in the present apparatus 100 may be realized by the CPU 410 executing a program that corresponds to each means and is stored in the ROM 420 or the auxiliary memory 440. Instead, each means included in the present apparatus 100 may be realized by hardware that performs processing related to each means. Further, the present apparatus 100 may be configured to execute a program according to the present invention by reading the program from an external server device through the communication I/F 450 or by reading the program according to the present invention from the recording medium 490 through the recording medium I/F 480.

### (Processing Example Executed by the Step-Count Management Apparatus According to the Present Embodiment)

### (1) Processing Example of Computing First Information 270 Executed by Present Apparatus 100

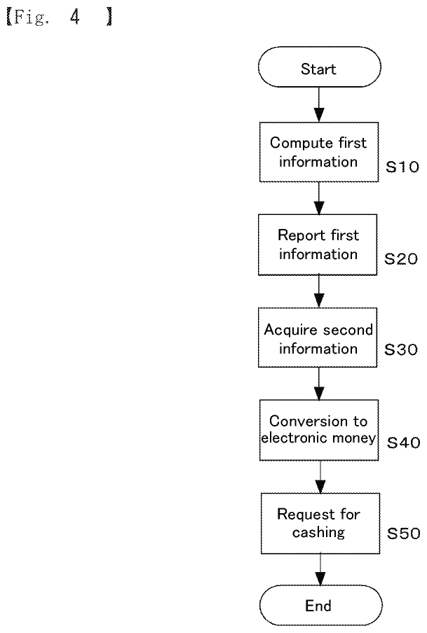

A processing example in which the present apparatus 100 computes the first information 270 is described with reference to FIG. 4. FIG. 4 is a flowchart illustrating the flow of a processing example, which is executed by the present apparatus 100 to compute the first information 270.

In step S10, the first information computation means 130 multiplies the number of steps 220 by the first coefficient 290 to thus compute the first information 270 that indicates a medical cost reduction effect commensurate with the number of steps 220 the user 210 makes. The medical cost reduction effect per step per day, for example, can be calculated as 0.065 to 0.072 Yen, which has been publicized by the Ministry of Land, Infrastructure, Transport and Tourism, on http://www.mlit.go.jp/report/press/toshi07_hh_000107.html.

In addition, in step S10, the first information computation means 130 may multiply the number of steps 220 corrected by the measurement environment identification information 320 by the first coefficient 290 to compute the first information 270. The correction based on the measurement environment identification information 320 includes correcting the number of steps 220 to become greater as the tilt of the ground increases from the viewpoint of exercise intensity, correcting the number of steps 220 to become smaller as the number of repetitive actions increases, and correcting the number of steps to become greater as exercise intensity increases. This is because it can be assumed that the medical cost reduction effect increases, as the exercise intensity of the user 210 becomes greater. In step S20, the first information reporting means 140 reports the first information 270 computed in step S10 to the health insurance server device 260 that is managed by the organization 340.

Then, the health insurance server device 260 multiplies the first information 270 reported in step S20 by the second coefficient 300 to thus compute the second information 280. It should be noted that the content of the second coefficient 300 used here is different from that of the second coefficient 300 in the processing example (2) that does not use the first information. 270, which is described later. The second coefficient 300 according to the present processing example is a coefficient for computing the second information 280 that can be cashed by the organization 340 on the basis of the first information 270. In step S30, the second information acquisition means 160 acquires from the health insurance server device 260 the second information 280 computed by the health insurance serer device 260.

In step S40, the electronic money conversation means 170 adds an amount of money computed by applying the second information acquired in step S30 by the third coefficient 310 to the balance 250 of the electronic money 240. The third coefficient 310 may be determined by considering the profit of the operator of the present apparatus 100.

In step S50, the cashing request means 180 requests the health insurance server device 260 to cash the second information 280 that has been converted into the electronic money 240. This is a process for making up for the amount of money that has been paid out to the user 210 as the electronic money 240 by the operator of the present apparatus 100.

As described above, the present apparatus 100 provides the user 210 with the electronic money 240 on the basis of the amount of walking the user 210 does. That is, the user 210 can obtain the electronic money 240 on the basis of the act of walking, and as a result, the user 210 is highly motivated for the act of walking. Meanwhile, as it has already been indicated that the act of walking has some effectiveness in reducing medical costs, the present apparatus 100 allows the user 210 who performs the act of walking to obtain a certain amount of money commensurate with the number of steps, thereby motivating the user 210 to perform the act of walking and facilitating medical cost. reduction.

### (2) Processing Example Executed by Present Apparatus 100 Without use of First Information 270

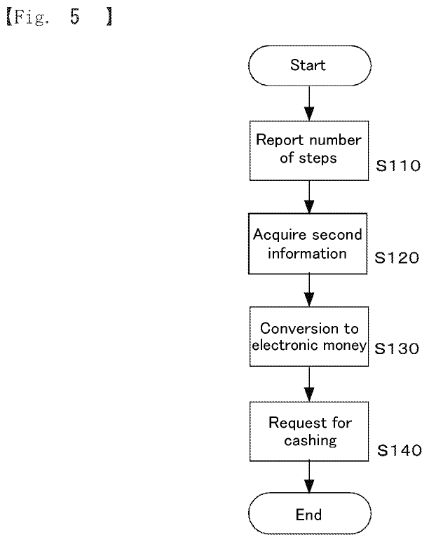

The processing example executed by the present apparatus 100 without use of the first information 270 is described with reference to FIG. 5. FIG. 5 is a flowchart illustrating the flow of a processing example executed by the present apparatus 100 without use of the first information 270.

In step S110, the step-count reporting means 150 reports the number of steps 220 to the health insurance server device 260. Additionally, the step-count reporting means 150 may have a configuration in which the number of steps 220 corrected based on the measurement environment identification information 320 is reported to the health insurance server device 260.

Here, the health insurance server device 260 multiplies the number of steps 220 reported in step S110 by the second coefficient 300 to thus compute the second information 280. It should be noted that the content of the second coefficient 300 used here is different from that of the second coefficient 300 in the processing example (1) for computing the first information 270. The second coefficient 300 according to the present processing example is a coefficient for computing the second information 280 that can be cashed by the organization 340 on the basis of the number of steps 220.

In step S120, the second information acquisition means 160 acquires from the health insurance server device 260 the second information 280 computed by the health insurance serer device 260.

In step 130, the electronic money conversion means 170 adds an amount of money that is computed by multiplying the second information acquired in step S120 by the third coefficient 310 to the balance 250 of the electronic money 240. The third coefficient 310 may be determined by considering the profit of the operator of the present apparatus 100.

In step S140, the cashing request means 180 requests the health insurance server device 260 to cash the second information 280 that has been converted into the electronic money 240. This is a process for making up for the amount of money that has been paid out to the user 210 as the electronic money 240 by the operator of the present apparatus 100.

As described above, the present apparatus 100 provides the user 210 with the electronic money 240 on the basis of the amount of walking the user 210 does. That is, the user 210 can obtain the electronic money 240 on the basis of the act of walking, and as a result the user 210 is highly motivated for the act of walking. Meanwhile, as it has already been indicated that the act of walking has some effectiveness in reducing medical costs, the present apparatus 100 allows the user 210 who performs the act of walking to obtain a certain amount of money commensurate with the number of steps, thereby motivating the user 210 to perform the act of walking and facilitating medical cost reduction.

While an embodiment according to the present invention has been described in detail above, the present invention is not limited to the specific embodiment, and various modifications and alterations may be made without departing from the scope of the present invention as defined in the appended claims.

### REFERENCE NUMERALS

100 Step-count management apparatus

110 Step-count memory

120 Electronic money balance memory

130 First information computation means

140 First information reporting means

150 Step-count reporting means

160 Second information acquisition means

170 Electronic money conversion means

180 Cashing request means

210 User

220 Number of steps (Amount of walking)

230 Pedometer

240 Electronic money

250 Balance of electronic money

260 Health insurance server device

270 First information

280 Second information

290 First coefficient

300 Second coefficient

310 Third coefficient

320 Measurement environment identification information

330 Communication network

340 Organization that provides insurance benefits covered by public medical insurance

410 CPU

420 ROM

430 RAM

440 Auxiliary memory

450 Communication interface

460 Input device

470 Output device (Display)

480 Recording medium interface

490 Recording medium

1. A step-count management apparatus comprising:
a step-count memory configured to store the number of steps measured by a pedometer that counts the number of steps a user makes;
an electronic money balance memory configured to store the balance of electronic money owned by the user;
a first information computation means configured to compute first information by multiplying the number of steps by a first coefficient that indicates a medical cost reduction effect per step, the first information indicating a medical cost reduction effect based on the number of steps the user makes;
a first information reporting means configured to report the first information to a health insurance server device that is managed by an organization that provides insurance benefits covered by public medical insurance;
a second information acquisition means configured to acquire, from the health insurance server device, second information that can be cashed by the organization, the health insurance server device multiplying the first information by a second coefficient to compute the second information based on the medical costs expected to be reduced due to the user's act of walking;
an electronic money conversion means configured to add an amount of money that is computed by multiplying the second information by a third coefficient for converting the second information into electronic money, to the balance of electronic money; and
a cashing request means configured to request the health insurance server device to cash the second information that has been converted into electronic money.

2. A step-count management apparatus comprising:
a step-count memory configured to store the number of steps measured by a pedometer that counts the number of steps a user makes;
an electronic money balance memory configured to store the balance of electronic money owned by the user;
a step-count reporting means configured to report the number of steps to a health insurance server device that is managed by an organization that provides insurance benefits covered by public medical insurance;
a second information acquisition means configured to acquire, from the health insurance server device, second information that can be cashed by the organization, the health insurance server device multiplying the number of steps by a second coefficient to compute the second information based on the medical costs expected to be reduced due to the user's act of walking;
an electronic money conversion means configured to add an amount of money that is computed by multiplying the second information by a third coefficient for converting the second information into electronic money, to the balance of electronic money; and
a cashing request means configured to request the health insurance server device to cash the second information that has been converted into electronic money.

3. The step-count management apparatus according to claim 1,
wherein the step-count memory associates and stores the number of steps and measurement environment identification information that is detected when the pedometer measures the number of steps, the measurement environment identification information indicating the environment in which the number of steps is measured, and
the first information computation means computes the first information by multiplying the number of steps corrected based on the measurement environment identification information by the first coefficient.

4. The step-count management apparatus according to claim 2,
wherein the step-count memory associates and stores the number of steps and measurement environment identification information that is detected when the pedometer measures the number of steps, the measurement environment identification information indicating the environment in which the number of steps is measured, and
the step-count reporting means reports the number of steps corrected based on the measurement environment identification information.

5. The step-count management apparatus according to claim 3, wherein the measurement environment identification information is made up of any one or more pieces of information that include the tilt of the ground where the user is located, the feature of action taken by the user such as the number of repetitive actions, a type of shoes worn by the user, and the strength of exercise taken by the user.

6. A step-count management method executed by a computer that includes a step-count memory for storing the number of steps measured by a pedometer that counts the number of steps a user makes and an electronic money balance memory for storing the balance of electronic money owned by the user, the step-count management method comprising:
a step in which a first information computation means computes first information that indicates a medical cost reduction effect based on the number of steps the user makes by multiplying the number of steps by a first coefficient that indicates a medical cost reduction effect per step;
a step in which a first information reporting means reports the first information to a health insurance server device that is managed by an organization that provides insurance benefits covered by public medical insurance;
a step in which a second information acquisition means acquires, from the health insurance server device, second information that can be cashed by the organization, the health insurance server device multiplying the first information by a second coefficient to compute the second information based on the medical costs expected to be reduced due to the user's act of walking;
a step in which an electronic money conversion means adds an amount of money that is computed by multiplying the second information by a third coefficient for converting the second information into electronic money, to the balance of electronic money; and
a step in which a cashing request means requests the health insurance server device to cash the second information that has been converted into electronic money.

7. A step-count management method executed by a computer that includes a step-count memory for storing the number of steps measured by a pedometer that counts the number of steps a user makes and an electronic money balance memory for storing the balance of electronic money owned by the user, the step-count management method comprising:
a step in which a step-count reporting means reports the number of steps to a health insurance server device that is managed by an organization that provides insurance benefits covered by public medical insurance;
a step in which a second information acquisition means acquires, from the health insurance server device, second information that can be cashed by the organization, the health insurance server device multiplying the number of steps by a second coefficient to compute the second information based on the medical costs expected to be reduced due to the user's act of walking;
a step in which an electronic money conversion means adds an amount of money that is computed by multiplying the second information by a third coefficient for converting the second information into electronic money, to the balance of electronic money; and
a step in which a cashing request means requests the health insurance server device to cash the second information that has been converted into electronic money.

8. The step-count management method according to claim 6, wherein the step-count memory associates and stores the number of steps and measurement environment identification information that is detected when the pedometer measures the number of steps, the measurement environment identification information indicating the environment in which the number of steps is measured, and the first information computation means computes the first information by multiplying the number of steps corrected based on the measurement environment identification information by the first coefficient.

9. The step-count management method according to claim 7, wherein the step-count memory associates and stores the number of steps and measurement environment identification information that is detected when the pedometer measures the number of steps, the measurement environment identification information indicating the environment in which the number of steps is measured, and the step-count reporting means reports the number of steps corrected based on the measurement environment identification information.

10. The step-count management method according to claim 8 or 9, wherein the measurement environment identification information is made up of any one or more pieces of information that include the tilt of the ground where the user is located, the feature of action taken by the user such as the number of repetitive actions, a type of shoes worn by the user, and the strength of exercise taken by the user.

11. A step-count management program for causing a computer to execute any one of methods according to claim 6.
